(19) United States
(12) Patent Application Publication
(10) Pub. No.: US 2022/0065787 A1
(43) Pub. Date: Mar. 3, 2022

(54) METHODS OF MFISH USING ALIGNMENT BEADS

(71) Applicant: Applied Materials, Inc., Santa Clara, CA (US)

(72) Inventors: Chloe Kim, San Jose, CA (US); Lu Yan, Cupertino, CA (US)

(21) Appl. No.: 17/460,052

(22) Filed: Aug. 27, 2021

Related U.S. Application Data
(60) Provisional application No. 63/072,894, filed on Aug. 31, 2020.

(51) Int. Cl. G01N 21/64; C12Q 1/6837; C12Q 1/6874

(52) U.S. Cl. CPC G01N 21/6456; C12Q 1/6837; G01N 2021/6441; G01N 2021/6419; G01N 2021/6421; C12Q 1/6874

(57) ABSTRACT

This disclosure relates to methods for image registration in fluorescence assays for the detection and quantitation of analytes in a sample using functionalized alignment beads as fiducial markers to improve image registration.

### CROSS-REFERENCE TO RELATED APPLICATIONS

This application claims priority to Provisional U.S. Application Ser. No. 63/072,894, filed on Aug. 31, 2020, the disclosure of which is incorporated by reference.

### TECHNICAL FIELD

This disclosure relates to methods for image registration in fluorescence assays for the detection and quantitation of analytes in a sample using functionalized alignment beads as fiducial markers to improve image registration.

### BACKGROUND

Fluorescence in situ hybridization (FISH) assays are a molecular cytogenetic method used in the detection and quantification of the presence or absence of specific nucleic acid (DNA or RNA) sequences in a sample, commonly used as a diagnostic tool in medicine and research. For example, FISH is used to detect the presence of chromosomal aberrations (gene mutations), changes in the expression profile and location of genes associated with different disease states (e.g. cancer, autoimmune disorders, psychiatric disorders, etc.), and species identification.

FISH involves the use of fluorescence microscopy, where the sample of interest is labeled with nucleic acid probes with high complementarity to bind to the genetic sequence of interest. These probes, or separate readout probes that bind to the nucleic acid probes, are labeled with a fluorophore, that when excited by an excitation source (such as from a fluorescent microscopy apparatus), emits a fluorescent signal. The signal is then detected and processed to convert the signals into an optical image, which typically depicts the spatial location and/or abundance of the analyte in the sample.

In particular, as part of FISH imaging, a sample is exposed to multiple oligonucleotide probes. During a round of hybridization, different oligonucleotide probes target different nucleotide sequences. Then a round of fluorescence images can be acquired by sequentially exposing the sample to excitation light of different wavelengths to excite the different probes. In addition, after a round of images is obtained, the probes can be photobleached, and another round of hybridization can be performed using more oligonucleotide probes that target still different nucleotide sequences, followed by another round of imaging.

For each given pixel, its fluorescence intensities from the different images form a signal sequence. This sequence is then compared to a library of reference codes from a codebook that associates each code with a gene. The best matching reference code can be used to identify an associated gene that is expressed at that pixel in the image.

### SUMMARY

In some implementations, the methods, systems, and fiducial markers described in this document can enable on-the-fly monitoring of mFISH performance, optimization of mFISH performance, or both. For example, mFISH performance can depend on various experimental settings that include, but are not limited to, control of fluidics for adequate buffer exchanges, on-stage flowcell hybridization, fluorescence intensities, photostability, focal plane, and/or image acquisition configurations. Use of the fiducial markers described in this document can enable monitoring, optimization, or both, of one or more of these configurations compared to prior systems, markers, or methods. In some examples, signals on these fiducial markers can indicate that the instrument or assay is performing appropriately.

On general, in one aspect, a method for performing an in situ fluorescence hybridization assay on a sample includes contacting the sample with one or more targeting-probes that bind to an analyte in the sample, if present, contacting the sample with a plurality of fiducial markers, that include a plurality of binding sites, contacting the sample with one or more readout-probes, with each readout-probe independently including a fluorescent moiety each readout probe binding with the one or more targeting probes, if present, and the plurality of binding sites, thereby exhibiting one or more fluorescent signals, imaging the one or more fluorescence signals produced by each readout-probe, and registering the image.

In general, another aspect of the subject matter described in this specification can be embodied in methods that include the actions of receiving a biological sample on a support, the sample having a first nucleotide sequence and a second nucleotide sequence or having a first and second pluralities of targeting probes that bind to targeted nucleotide sequences on the sample with the first plurality of targeting probes having the first nucleotide sequence and the second plurality of targeting probes having the second nucleotide sequence; receiving a plurality of beads on the support, each bead having a plurality of binding sites, a first subset of the plurality of binding sites including the first nucleotide sequence and a second subset of the plurality of binding sites the second nucleotide sequence; exposing the sample and plurality of beads to a first plurality of first probes, each first probe having a fluorophore and a complementary first nucleotide sequence such that the complementary first nucleotide sequence binds to the first nucleotide sequences on the beads and first nucleotide sequences in the sample or in the first plurality of targeting probes; obtaining a first image of the sample and the plurality of beads; deactivating the first plurality of first probes; subjecting the sample and plurality of beads with a second plurality of second probes, each second probe having a fluorophore and a complementary second nucleotide sequence such that the complementary second nucleotide sequence binds to the second nucleotide sequence on the beads and the second nucleotide sequence in the sample or in the second plurality of targeting probes; obtaining a second image of the sample and the plurality of beads; detecting locations of the plurality of beads in the first image and the second image; and performing a registration of the first image and the second image based on the detected locations.

Other embodiments of this aspect include corresponding computer systems, apparatus, computer program products, and computer programs recorded on one or more computer storage devices, each configured to perform the actions of the methods. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The foregoing and other embodiments can each optionally include one or more of the following features, alone or in combination.

The method can include repeating steps of contacting through imaging from 0 to 10 times. The method can include photobleaching the sample, and independently repeating the step of contacting through photobleaching from 0 to 10 times, e.g., 1-9 times providing 2-10 total rounds of imaging, e.g., 1-3 times, thereby providing 2-4 total rounds of imaging. The sample may be washed between contacting the sample with one or more targeting-probes and contacting the sample with the plurality of fiducial markers, or between contacting the sample with the plurality of fiducial markers and contacting the sample with one or more readout-probes, or between contacting the sample with one or more readout-probes and imaging.

The analyte in the sample, if present, may include an oligonucleotide. The one or more targeting-probes may include an oligonucleotide. Each readout probe may further include an oligonucleotide. Each readout probe may consist essentially of a fluorescent moiety and an oligonucleotide.

Each fluorescent moiety may independently be a fluorescent dye or a fluorescent polypeptide. Each fluorescent moiety may independently be a fluorescent dye. Each fluorescent moiety may be independently selected from the group consisting of: pacific blue (PacB), Horizon V450, pacific orange (PacO), aminomethylcoumarin acetate (AMCA), fluorescein isothiocyanate (FITC), Alexa488, phycoerythrin (PE), peridinin chlorophyl protein/cyanine 5.5 (PerCP-Cy5.5), PerCP, PE-TexasRed, phycoerythrin/cyanine7 (PE-Cy7), allophycocyanine (APC), Alexa594, cyanine 5.5 (Cy5.5), IR800, Alexa647, allophycocyanine/H7 (APC-H7), APC-Cy7, Alexa680, and Alexa700.

The plurality of fiducial markers may be beads, and the beads may include a bead core and a bead surface, wherein the surface comprises the plurality of binding sites. The bead core may include non-porous silica or an organic polymer. The bead core may include an organic polymer selected from polystyrene, polyisoprene, and latex. The diameter of the beads may be about 0.05 μm to about 1 μm. Each of the plurality of binding sites may include an oligonucleotide. Each oligonucleotide may independently be DNA or RNA. Each oligonucleotide may independently be DNA. Each oligonucleotide may independently be RNA. Each oligonucleotide may independently comprise 15 to 30 residues.

The sample may be from a human subject. The sample may be from a solid or liquid biopsy from a human subject. The human subject may have been previously diagnosed with cancer.

The method can include, for each of a plurality of pixels in the registered images, determining expression of genes in the sample based on intensities of fluorescence of the sample in the first image and the second image. Deactivating the first plurality of first probes can include photobleaching the first plurality of first probes. Deactivating the first plurality of first probes can include purging the first plurality of first probes.

In some implementations, the first image and the second image can have the same lateral position. The first image and the second image can have the same color channel. Obtaining the first image and obtaining the second image can include exciting the fluorophores in the first plurality of first probes and the second plurality of second probes with the same excitation wavelength. The fluorophores in the first plurality of first probes and the fluorophores of second plurality of second probes can have the same material composition. The sample can have a third nucleotide sequence and a fourth nucleotide sequence or can have a third and fourth pluralities of targeting probes that bind to targeted nucleotide sequences on the sample with the third plurality of targeting probes having the third nucleotide sequence and the fourth plurality of targeting probes having the fourth nucleotide sequence.

In some implementations, the method can include prior to deactivating the first plurality of first probes: exposing the sample and plurality of beads to a third plurality of third probes, each third probe having a fluorophore and a complementary third nucleotide sequence such that the complementary third nucleotide sequence binds to the third nucleotide sequences on the beads and the third nucleotide sequences in the sample or in the third plurality of targeting probes, and obtaining a third image of the sample and the plurality of beads. The first image and the third image can use different color channels. Fluorophores of the first plurality of first probes and fluorophores of the third plurality of third probes can emit at different wavelengths. Fluorophores of the first plurality of first probes and fluorophores of the third plurality of third probes can be excited by different excitation wavelengths. The method can include detecting locations of the plurality of beads in the third image, and performing a registration of the first image and the third image based on the detected locations.

In some implementations, the method can include deactivating the third plurality of third probes; after deactivating the first plurality of first probes and the third plurality third probes: subjecting the sample and plurality of beads with a fourth plurality of fourth probes, each fourth probe having a fluorophore and a complementary fourth nucleotide sequence such that the complementary fourth nucleotide sequence binds to the fourth nucleotide sequence on the beads and the fourth nucleotide sequence in the sample or in the fourth plurality of targeting probes; and obtaining a fourth image of the sample and the plurality of beads. The second image and the fourth image can use different color channels. The third image and the fourth image can use the same color channel.

In some implementations, fluorophores of the second plurality of second probes and fluorophores of the fourth plurality of fourth probes can emit at different wavelengths. Fluorophores of the third plurality of third probes and fluorophores of the fourth plurality of fourth probes can emit at the same wavelength. The fluorophores in the third plurality of third probes and the fluorophores of fourth plurality of fourth probes can have the same material composition. Fluorophores of the second plurality of first probes and fluorophores of the fourth plurality of fourth probes can be excited by different excitation wavelengths. Fluorophores of the third plurality of third probes and fluorophores of the fourth plurality of fourth probes can be excited by the same excitation wavelength. The method can include detecting locations of the plurality of beads in the fourth image, and performing a registration of the first image and the fourth image based on the detected locations.

Another aspect of the subject matter described in this specification can be embodied in an article of manufacturing that includes a plurality of alignment beads, each alignment bead having a plurality of binding sites, the plurality binding sites including a first subset binding sites that include a first nucleotide sequence, wherein the first nucleotide sequence is selected to bind to a complementary first nucleotide sequence of a first probe that has a fluorophore and that targets the first nucleotide sequence in a sample or in a first targeting probe that targets the sample; a second subset of the plurality of binding sites that include a second nucleotide sequence, wherein the second nucleotide sequence is selected to bind to a complementary second nucleotide sequence of a second probe that has a fluorophore and that targets the second nucleotide sequence in a sample or in a second targeting probe that targets the sample, wherein the fluorophore of the first probe has a same emission wavelength and/or same excitation wavelength as the fluorophore of the second probe.

In some embodiments, the plurality binding sites include a third subset binding sites that include a third nucleotide sequence, wherein the third nucleotide sequence is selected to bind to a complementary third nucleotide sequence of a third probe that has a fluorophore and that targets the third nucleotide sequence in the sample or in a third targeting probe that targets the sample, wherein the fluorophore of the third probe has a different emission wavelength and/or different excitation wavelength as the fluorophore of the first probe.

In some embodiments, the plurality binding sites include a fourth subset binding sites that include a fourth nucleotide sequence, wherein the fourth nucleotide sequence is selected to bind to a complementary fourth nucleotide sequence of a fourth probe that has a fluorophore and that targets the fourth nucleotide sequence in the sample or in a fourth targeting probe that targets the sample, wherein the fluorophore of the fourth probe has a same emission wavelength and/or same excitation wavelength as the fluorophore of the third probe.

The subject matter described may result in, but is not limited to, one or more of the following advantages.

Different color channels can have higher accuracy registration by registering images. The higher accuracy can result from capture of a single image that depicts data for both the sample and the fiducial marker, which can compensate for movement of the sample, the fiducial marker, or both, in a liquid that can occur during a time between capture of separate images for the sample and the fiducial marker.

Computing resources required to perform mFISH can be reduced. For instance, an mFISH apparatus can use fewer computer cycles, e.g., be faster, compared to other systems because it captures a single image that depicts data for both a sample and a fiducial marker rather than two images, one for the sample and another for a fiducial marker. An mFISH apparatus can use fewer computer cycles compared to other systems because it analyzes fewer images during a registration process, a stitching process, or both. In some examples, an mFISH apparatus can use less memory compared to other systems because it can store a single image that depicts data for a sample and a fiducial marker instead of two images.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

FIG. 1 depicts an alignment bead functionalized with nine different binding sites and labeled with three probes of different color dyes.

FIG. 2 are representative fluorescent microscopy images of a sample sequentially labeled with six different probes each of a single color using a 1-μm diameter alignment bead as fiducial marker.

FIG. 3 are representative fluorescent microscopy images acquired sequentially of a 1-μm diameter alignment bead labeled with three probes for five rounds of hybridization.

FIG. 4 are representative fluorescent microscopy images acquired sequentially of a 200-nm diameter alignment bead labeled with three probes for six rounds of hybridization.

FIG. 5 depicts a multiplexed fluorescent in-situ hybridization (mFISH) imaging and image processing apparatus.

FIG. 6 is a flow diagram of a process for registering a first image and a second image.

FIG. 7 illustrates a flow chart of a process of data processing in which the processing is performed after images have been acquired.

### DETAILED DESCRIPTION

During the FISH imaging process, an image processing apparatus can capture images of a certain portion of a sample at multiple different times. For example, images of a portion of a sample can be captured at different times for different probes, e.g., for different excitation wavelengths within a round of hybridization or for different rounds of hybridization.

An issue is that in some situations, the carrier material on the microscope slide can move or samples desired to be imaged can move within the carrier material. For example, samples suspended in a liquid could move. In this case, even if the stage supporting the slide is moved accurately to the same position, the sample may no longer be in the same location in the later image, which can make comparison of the images of the sample difficult. A technique to compensate for this motion is to place fiducial markers, e.g., fluorescent beads, within the carrier material on the slide. In general, the sample and the fiducial marker beads will move approximately in unison. By comparison of the positions of the fiducial markers permits registration of the two images.

In conventional systems, the sample and the fiducial markers used to perform the registration have different fluorescence colors. Thus, two fluorescent microscope images are captured: one depicting only the sample (i.e., with the first fluorescence color), and one depicting only the fiducial markers (i.e., with the second fluorescence color). A problem is that even in this situation, some movement of the sample can occur between the time that the image of the sample and the image of the fiducial markers is taken. Hypothetically a fiducial bead to which fluorophores are attached that emit the same color as a probe. But this provides registration for only a single color channel, e.g., different hybridization steps, not between different color channels.

A related issue is that the FISH system may image multiple different regions of a sample, e.g., that is on a slide. The FISH system can capture these images by moving the sample along an x-axis, a y-axis, or both. The image processing apparatus can place the sample at a first location under a microscope, e.g., a fluorescence microscope, and capture a first image of a first field of view (“FOV”) under the microscope. The image processing apparatus can then adjust an x-coordinate, a y-coordinate, or both, of the sample with respect to the microscope to place the sample at a second location under the microscope. The image processing apparatus can capture a second image of a second FOV under the microscope.

A system can combine data for multiple images, e.g., multiple FOVs, of the sample to create a single image of the sample. For instance, the system can align fiducial markers, e.g., fluorescent beads in overlapping portions of the images, to create the single image of the sample. As discussed above, when the image processing apparatus captures separate images of the sample and the fiducial markers, there can be drift between the capture of these two images.

Yet another issue is that different properties of different fluorophores can lead to unbalanced signal intensities in this analysis, which provides an inaccurate representation of the abundance and/or location of the analytes. In a clinical setting, this could mean the different between a patient being diagnosed with cancer, or given a clean bill of health.

Attempts to account for this problem using conventional fiducial markers have generally been unsuccessful, at least because these markers only use a single fluorophore, which loses intensity during rounds imaging. Similarly, different sets of markers, each with a single fluorophore, still loses signal intensity at different rates.

To account for movement of the sample across images, an image processing apparatus, e.g., an mFISH apparatus, can capture a single image of a FOV that includes data for both the sample and the fiducial marker. To enable capture of a single image that includes data for both the sample and the fiducial marker, the image processing apparatus can use fiducial markers with binding sites that are the same as the binding sites on the targeting-probes that bind to analytes, e.g., substances, in the sample. The substances can be any appropriate substances that are being analyzed, such as DNA or RNA. The image processing apparatus can introduce readout-probes into the fluid that bind to both targeting-probes and fiducial markers because of the common binding sites. The readout-probes can be for a particular wavelength or range of wavelengths and enable capture of image data for the analytes.

Additionally, the fiducial markers can include multiple binding sites for different readout-probes that illuminate at different wavelengths. This can enable the image processing apparatus to capture images at different wavelengths using the same fiducial markers rather than using different fiducial markers for different wavelengths which might not be at the same location in a sample.

### Definitions

As used herein, the term “about” is used to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “each,” when used in reference to a collection of items, is intended to identify an individual item in the collection but does not necessarily refer to every item in the collection, unless expressly stated otherwise, or unless the context of the usage clearly indicates otherwise.

### Overview

Some embodiments provide a method for performing an in situ fluorescence hybridization assay on a sample, the method comprising:

a) Contacting the sample with one or more targeting-probes, wherein each targeting-probe binds to an analyte in the sample, if present;

b) Contacting the sample with a plurality of fiducial markers, wherein each fiducial marker comprises a plurality of binding sites;

c) Contacting the sample with one or more readout-probes, wherein each readout-probe independently comprises a fluorescent moiety, and wherein each readout probe binds with the one or more targeting probes, if present, and the plurality of binding sites, thereby exhibiting one or more fluorescent signals;

d) Imaging the one or more fluorescence signals produced by each readout-probe;

e) Registering the image in step d);

f) Repeating steps (a)-(e) from 0 to 10 times;

g) Photobleaching the sample; and

h) Independently repeating steps (a)-(g) from 0 to 10 times.

In some embodiments, the method further comprises one or more washing steps. For example, in some embodiments, the sample is washed between steps 1(a) and 1(b), between steps 1(b) and 1(c), between steps 1(c) and 1(d), or a combination of any of the foregoing. In some embodiments, steps (a)-(e) are performed in the order in which they are recited. In some embodiments, one or more of steps (a)-(e) are performed in a different order than recited.

In some embodiments, step (h) is repeated from 1-9 times, thereby providing 2-10 total rounds of imaging. For example, step (h) may be repeated 1 time, 2 times, 3 times, 4 times, 5 times, 6 times, 7 times, 8 times, 9 times, or 10 times, thereby providing 2, 3, 4, 5, 6, 7, 8, 9, 10, or 11 rounds of imaging, respectively. In some embodiments, step (h) is repeated from 1-4 times, thereby providing 2-5 total rounds of imaging. In some embodiments, step (h) is repeated once. In some embodiments, steps step (h) is are repeated twice. The phrase “rounds of imaging” refers to the total number of times step (h) is performed (the number of times steps (a)-(g) are repeated), for example, when the steps are performed once, one round of imaging is performed.

In some embodiments, step (f) is repeated from 1-9 times, wherein each of the targeting-probes, fiducial markers, binding sites, readout-probes, and fluorescent moieties can each be independently the same or different in each repetition of step (f). For example, different fluorescent moieties may be used to general different fluorescent signals.

In some embodiments, the fiducial markers comprise oligonucleotide-functionalized alignment beads. In some embodiments, single-color probes are used to label both in a single round of hybridization experiment, where the probes are designed to bind to both the analytes (such as nucleic acid molecules) and to the binding sites of the alignment beads (such as oligonucleotide binding sites). In some embodiments, multiple-color probes designed to hybridize to both the analytes and the binding sites of the alignment beads, as described herein, are used to label both in a single round of hybridization experiment. In some embodiments, a fiducial identification system can be used to identify alignment beads in a fluorescence microscopy image of a microscope slide comprising alignment beads and a sample, each having a common fluorescence wavelength. The fluorescence microscopy image depicts both the fiducial markers and the sample. In some embodiments, registering the image comprises a fiducial identification system capable of registering fluorescence images captured at different time points.

In some embodiments, the analyte is absent from the sample. Thus, the targeting probes will bind solely to the binding sites on the fiducial markers, and the resulting fluorescent signal from the readout probe will indicate only the presence of the fiducial markers, demonstrating that the analyte is absent in the sample.

It is to be understood that the fluorescent moiety does not exhibit a fluorescent signal based on a binding interaction, such as a readout-probe binding to targeting probe or a binding site on a fiducial marker. Rather, the fluorescent moiety exhibits a fluorescent signal after excitation with a photon of an appropriate wavelength, for example, from a fluorescence microscopy apparatus.

In some embodiments, “binding” comprises hybridization. For example, the binding of the targeting-probes to the analyte, the targeting-probes to the binding sites on the fiducial markers, and the readout probes to the targeting-probes can each independently comprise hybridization. In some embodiments “binding” is hybridization of oligonucleotides.

In some embodiments, the plurality of binding sites are the same. In some embodiments, the plurality of binding sites are different from one another. In some embodiments, each bead can have from 30 to 100 different binding sites. In some embodiments, each bead can have from 1 to 30 different binding sites. In some embodiments, the plurality of binding sites comprises from 1 to 100 different binding sites, such as, 1 to 30 binding sites, 1 to 10 binding sites, 5 to 15 binding sites, or 10 to 20 binding sites. In some embodiments, the plurality of binding sites is 1, 2, 3, 4, 5, 6, 7, 8, 9, 10, 11, 12, 13, 14, 15, 16, 17, 18, 19, or 20 different binding sites. In some embodiments, the plurality of binding sites is 4, 5, 6, 7, 8, 9, or 10 different binding sites.

In some embodiments, the density of binding sites on the alignment beads is about 480 to about 2400 per bead. In some embodiments, the density of binding sites on the alignment bead is about 480 to about 1200 per bead, about 1200 to about 2400 per bead, about 1200 to about 1800 per bead, about 720 to about 1200 per bead, or about 960 to about 1800 per bead. In some embodiments, the density of binding sites on the alignment beads is about 1200 per bead.

In some embodiments, the analyte in the sample, if present, comprises an oligonucleotide. In some embodiments, the analyte in the sample comprises an oligonucleotide that is longer than 15 bp. In some embodiments, the analyte in the sample comprises an oligonucleotide that is about 15 bp to about 1000 bp long. In some embodiments, each of the one or more targeting-probes comprises an oligonucleotide. In some embodiments, each of each readout probe further comprises an oligonucleotide. In some embodiments, each readout probe consists essentially of a fluorescent moiety and an oligonucleotide.

A “fluorescent moiety” refers to a substance that emits light of a particular wavelength (e.g., having a particular color) in response to being exposed to a light of a wavelength that “excites” the fluorescent material. A fluorescent material can be coupled to other molecules (e.g., DNA molecules, proteins, antibodies, or beads). For example, a fluorescent material can be coupled to a DNA molecule using a NHS ester-type reaction. A fluorescence microscope is an optical microscope that generates an image by exposing a fluorescent material to light of a wavelength that excites the fluorescent material. Exemplary fluorescent materials include pacific blue (PacB), Horizon V450, pacific orange (PacO), aminomethylcoumarin acetate (AMCA), fluorescein isothiocyanate (FITC), Alexa488, phycoerythrin (PE), peridinin chlorophyl protein/cyanine 5.5 (PerCP-Cy5.5), PerCP, PE-TexasRed, phycoerythrin/cyanine7 (PE-Cy7), allophycocyanine (APC), Alexa594, cyanine 5.5 (Cy5.5), IR800, Alexa647, allophycocyanine/H7 (APC-H7), APC-Cy7, Alexa680 and Alexa700.

In some embodiments, each fluorescent moiety is independently a fluorescent dye or a fluorescent polypeptide. In some embodiments, each fluorescent moiety is independently a fluorescent dye. In some embodiments, each fluorescent moiety is independently selected from the group consisting of: pacific blue (PacB), Horizon V450, pacific orange (PacO), aminomethylcoumarin acetate (AMCA), fluorescein isothiocyanate (FITC), Alexa488, phycoerythrin (PE), peridinin chlorophyl protein/cyanine 5.5 (PerCP-Cy5.5), PerCP, PE-TexasRed, phycoerythrin/cyanine7 (PE-Cy7), allophycocyanine (APC), Alexa594, cyanine 5.5 (Cy5.5), IR800, Alexa647, allophycocyanine/H7 (APC-H7), APC-Cy7, Alexa680, and Alexa700.

In some embodiments, each of the plurality of binding sites comprises an oligonucleotide. In some embodiments, each oligonucleotide is independently DNA or RNA. In some embodiments, each oligonucleotide is independently DNA. In some embodiments, each oligonucleotide is independently RNA. In some embodiments, each oligonucleotide independently comprises 15-30 residues.

Some embodiments provide a method for performing an in situ fluorescence hybridization assay on a sample, the method comprising:

a) Contacting the sample with one or more probes, wherein each probe comprises a targeting domain and a readout domain, wherein each readout domain exhibits a signal detectable by a fluorescence imaging system;

b) Contacting the sample with a plurality of fiducial markers, wherein each fiducial marker comprises a plurality of binding sites; wherein each targeting domain hybridizes to (i) an analyte in the sample, if present, thereby exhibiting a fluorescence signal, and (ii) a binding site of a fiducial marker, thereby exhibiting a fluorescence signal;

c) Imaging the fluorescence signals produced by each readout domain;

d) Registering the image in step c) and

e) Photobleaching the sample.

In some embodiments, steps (a)-(e) are performed in the order in which they are recited. In some embodiments, one or more of steps (a)-(e) are performed in a different order than recited.

In some embodiments, steps (a)-(e) are repeated from 1-9 times, thereby providing 2-10 total rounds of imaging. For example, steps (a)-(e) may be repeated 1 time, 2 times, 3 times, 4 times, 5 times, 6 times, 7 times, 8 times, 9 times, or 10 times, thereby providing 2, 3, 4, 5, 6, 7, 8, 9, 10, or 11 rounds of imaging, respectively. In some embodiments, steps (a)-(e) are repeated from 1-3 times, thereby providing 2-4 total rounds of imaging. In some embodiments, steps (a)-(e) are repeated once. In some embodiments, steps (a)-(e) are repeated twice. The phrase “rounds of imaging” refers to the total number of times steps (a)-(e) are performed, for example, when the steps are performed once, one round of imaging is performed.

In some embodiments, the method further comprises repeating a subset of steps (a)-(d) prior to step (e), for example, step (a), step (b), step (c), step (d), or any combination of the foregoing, may be repeated from 1-4 times prior to step (e).

In some embodiments, the plurality of binding sites comprises from 1 to 20 binding sites, for example, a first, second, third, a fourth, and so forth, binding site. In some embodiments, the plurality of binding sites comprises a first binding site and a second binding site.

In some embodiments, the one or more probes comprises a plurality of first, second, third, and/or fourth probes. In some embodiments, the first probes comprise a first targeting domain that hybridizes to a first binding site on the fiducial markers; the second probes comprise a second targeting domain that hybridizes to a second binding site on the fiducial markers; the third probes comprise a third targeting domain that hybridizes to a third binding site on the fiducial markers; and the fourth probes comprise a fourth targeting domain that hybridizes to a fourth binding site on the fiducial markers.

In some embodiments, the one or more probes comprises a plurality of first probes; wherein the first probes comprise a first targeting domain that hybridizes to a first binding site on the fiducial markers. In some embodiments, the one or more probes comprises a plurality of second probes; wherein the second probes comprise a second targeting domain that hybridizes to a second binding site on the fiducial markers.

In some embodiments, each targeting domain is different. In some embodiments, each targeting domain is the same. In some embodiments, the first targeting domain and the second targeting domain are different.

In some embodiments, the sample is contacted with the plurality of first, second, third, and/or fourth probes, respectively. In some embodiments, the sample is contacted with the plurality of first probes prior to the sample being contacted with the plurality of second probes.

In some embodiments, the sample is contacted with the plurality of first, second, third, and/or fourth probes in the same round of imaging. In some embodiments, the sample is contacted with the plurality of first, second, third, and/or fourth probes in two or more different rounds of imaging. In some embodiments, the sample is contacted with the plurality of first probes and the plurality of second probes in the same round of imaging. In some embodiments, the sample is contacted with the plurality of first probes and the plurality of second probes in the different rounds of imaging.

Photobleaching (also known as fading) of a fluorescence signal occurs when a fluorophore undergoes irreversible modifications to its chemical structure due to photon-induced chemical changes. Since different fluorophores have different chemical structures and fluorescence properties, the fluorescence signal intensities from each of the different fluorophores in a multi-color (multiplexed) FISH experiment may become substantially different after a period of light exposure using one or more excitation wavelengths depending on each fluorophore's intrinsic sensitivity to photobleaching. The differential sensitivity to photobleaching of different fluorophores can lead to unbalanced signal intensities in a multiplex FISH experiment, and consequentially, yielding an inaccurate representation of the relative abundance of different analytes labeled and detected by the different fluorescent probes used. Conventional fiducial markers use only a single fluorophore, which will lose intensity over time (such as over rounds of signaling). Even using different sets of beads, each with a single fluorophore, will lose signal intensity at different rates. Thus, conventional fiducial beads would not function as location markers, at least because the same beads within the same FOV in different registered images. There is no simple mechanism to restore the fluorescence signal on the marker to prevent unbalanced signal intensities in the image. This phenomenon is particularly problematic in a multiple-round imaging where the fluorescence signal intensities from fiducial markers labeled with different fluorophores can differ substantially throughout the different rounds of imaging.

In contrast, the fiducial markers, as described herein, can be labeled with one or more different types of fluorophores on the same bead in each round of hybridization, as described herein. For example, each targeting-probe comprises one oligonucleotide region that binds to the analyte in the sample (if present) and to the binding sites on the fiducial markers, and a second oligonucleotide region that binds to the readout-probe. Thus, in each round of imaging, targeting-probes having the same second oligonucleotide region (which will all bind to the same readout-probe) or having one or more different second oligonucleotide regions (which will each bind to a different readout-probe) may be used. In turn, each readout probe may comprise a different fluorescent moiety, thus facilitating imaging of multiple channels of color in each individual round of imaging and/or over multiple rounds of imaging.

In some embodiments, the beads are labeled with a plurality of first probes in the same round of hybridization. In some embodiments, the beads are labeled with a mixture of a plurality of first and second probes in the same round of hybridization. In some embodiments, the beads are labeled with a mixture of a plurality of first, second and third probes in the same round of hybridization. In some embodiments, the fiducial marker beads are labeled with a mixture of a plurality of first, second, third and fourth probes in the same round of hybridization. In some embodiments, all of the existing fluorescence signals from the first set of probes are intentionally removed by photobleaching and a new set of probes are added to the sample at the end of the first round of hybridization and imaging. In some embodiments, the hybridization/imaging/photobleaching steps are performed for one round. In some embodiments, the hybridization/imaging/photobleaching steps are repeated for 2-3 rounds. In some embodiments, the hybridization/imaging/photobleaching steps are repeated for 4-7 rounds. In some embodiments, the hybridization/imaging/photobleaching steps are repeated for 8-10 rounds.

In some embodiments, the same field-of-view (FOV) is imaged throughout the different rounds of imaging, wherein any shifts in the fluorescence signals from the fiducial marker beads and analytes in the sample can be tracked throughout. In some embodiments, the fluorescence signals from 3 fiducial marker beads within the same FOV is sufficient to enable correction of shifts between different imaging rounds. In some embodiments, the fluorescence signals from 5 fiducial marker beads within the same FOV is sufficient to enable correction of shifts between different imaging rounds. In some embodiments, the fluorescence signals from 10 fiducial marker beads within the same FOV is sufficient to enable correction of shifts between different imaging rounds. In some embodiments, the fluorescence signals from 20 fiducial marker beads within the same FOV is sufficient to enable correction of shifts between different imaging rounds.

In some embodiments, the plurality of fiducial markers comprise beads, wherein the beads comprise a bead core and a bead surface, wherein the surface comprises a plurality of binding sites. In some embodiments, the bead core comprises non-porous silica or an organic polymer. In some embodiments, the bead core comprises an organic polymer selected from polystyrene or polyisoprene.

In some embodiments, the diameter of the beads is about 0.050 μm to about 1 μm. In some embodiments, each readout domain of each probe independently comprises a fluorescent dye. In some embodiments, each fluorescent dye is independently selected from pacific blue (PacB), Horizon V450, pacific orange (PacO), aminomethylcoumarin acetate (AMCA), fluorescein isothiocyanate (FITC), Alexa488, phycoerythrin (PE), peridinin chlorophyl protein/cyanine 5.5 (PerCP-Cy5.5), PerCP, PE-TexasRed, phycoerythrin/cyanine7 (PE-Cy7), allophycocyanine (APC), Alexa594, cyanine 5.5 (Cy5.5), IR800, Alexa647, allophycocyanine/H7 (APC-H7), APC-Cy7, Alexa680, and Alexa700. In some embodiments, the readout domain of the probe comprises a fluorescent peptide or protein, such as GFP. In some embodiments, the readout domain of the probe comprises a quantum dot.

In some embodiments, the alignment beads are not auto-fluorescent. In some embodiments, the alignment beads are not auto-fluorescent in about the same wavelength as any readout domain. In some embodiments, alignment beads comprise non-porous silica. In some embodiments, the alignment beads comprise one or more organic polymers. Examples of such organic polymers include, but are not limited to, polystyrene, polyethylene, polypropylene, and poly(vinyl)alcohol. In some embodiments, the alignment comprise a dispersed colloidal suspension of spherical particles comprising amorphous polyisoprene (latex).

In some embodiments, the diameter of the alignment beads is about 0.05 micrometers to about 1 micrometers (μm). The beads can be equal to, or larger than, the pixel size of the image, e.g., 70-120 nm, but need not be larger than about 10 times the pixel size. In some embodiments, the diameter of the beads is about 0.2 μm. In some embodiments, the diameter of the beads is about the same as the diameter of the target spot, thereby having comparable signal intensity to that of the target spot. The signal intensity of the alignment beads can be adjusted to match with the signal intensity of the target region for balanced signal level by adjusting the density of binding sites on the alignment beads. The fluorescence intensity from the labeled beads can be adjusted to match with the fluorescence intensity of the target region for balanced signal level by adjusting the density of probes used to label the beads (i.e., less probes can be used for labeling larger beads). In some embodiments, the density of binding sites on the alignment beads is about 480 to about 2400. In some embodiments, the density of binding sites on the alignment beads is about 1200.

In some embodiments, each targeting domain comprises an oligonucleotide. In some embodiments, each binding site comprises an oligonucleotide. In some embodiments, each analyte comprises an oligonucleotide. In some embodiments, the oligonucleotide is DNA. In some embodiments, the oligonucleotide is RNA. In some embodiments, the each oligonucleotide independently comprises 15 to 30 residues.

Attachment of the oligonucleotide binding sites to the alignment beads can be achieved via appropriate chemical coupling reactions which are well known to those skilled in the art. In some embodiments, the chemical functional groups (coupling partners) for a covalent coupling reaction are amine/carboxyl groups in an amide-bond forming reaction. In some embodiments, the coupling partners for a coupling reaction are thiol/maleimide groups in a Michael reaction. In some embodiments, the coupling partners for a coupling reaction are thiol/disulfide groups in a disulfide exchange reaction. In some embodiments, the coupling partners for a coupling reaction are hydroxyl/epoxy groups in an epoxy ring-opening reaction. In some embodiments, the coupling partners for a coupling reaction are amino/epoxy groups in an epoxy ring-opening reaction.

The chemical functional group is linked to the 5′ phosphate group of the oligonucleotides. In some embodiments, the chemical functional group is linked to the 5′ phosphate group of the oligonucleotides with a spacer of about 3 to about 16 carbon atoms, for example, a 3 carbon spacer, a 4 carbon spacer, a 5 carbon spacer, a 6 carbon spacer, a 7 carbon spacer, a 8 carbon spacer, a 9 carbon spacer, a 10 carbon spacer, a 11 carbon spacer, a 12 carbon spacer, a 13 carbon spacer, a 14 carbon spacer, a 15 carbon spacer, or a 16 carbon spacer. In some embodiments, the spacer is a 6 carbon spacer. In some embodiments, the spacer is a 12 carbon spacer.

In some embodiments, the surface charges on the alignment beads can be positive or negative, depending on the identity of the functional groups present. In some embodiments, the alignment beads comprise one or positively charged groups impart positive surface charges to the beads, for example, one or more amidine groups. In some embodiments, the alignment beads comprise one or negatively charged groups impart negative surface charges to the beads, for example, one or more carboxyl and/or sulfate groups. In some embodiments, the alignment beads have no net surface charge, for example, no positively or negatively charged groups, or an equal number of positively and negatively charged groups resulting in no net surface charge.

In some embodiments, the surface charge densities on the alignment beads is about 70 Å per charge group to about 1000 Å per charge group, for example, about 70 Å to about 300 Å per charge group, about 200 Å to about 400 Å per charge group, about 300 Å to about 500 Å per charge group, about 400 Å to about 600 Å per charge group, about 600 Å to about 800 Å per charge group, about 800 Å to about 1,000 Å per charge group, or any value in between.

The chemical coupling reactions are carried out under appropriate reaction conditions (reaction solvent, additives, pH, temperature, reaction time) that favor the progression of the coupling reaction.

In some embodiments, the coupling reactions are carried out in an aqueous buffer. In some embodiments, the aqueous buffer has a pH between about 2 and about 10, for example, about 2, about 3, about 4, about 5, about 6, about 7, about 8, about 9, about 10, or any value in between.

In some embodiments, the aqueous buffer comprises (N-morpholino)ethanesulfonic acid (MES), tris(hydroxymethyl)aminomethane (Tris), or tris(hydroxymethyl)aminomethane-ethylenediaminetetraccetic acid (TE) buffer. In some embodiments, the aqueous buffer is (N-morpholino)ethanesulfonic acid (IVIES). In some embodiments, the buffer is tris(hydroxymethyl)aminomethane (Tris). In some embodiments, the buffer is tris(hydroxymethyl)aminomethane-ethylenediaminetetraccetic acid (TE) buffer.

In some embodiments, one or more additives are used to promote the coupling reaction. In some embodiments, the one or more additive comprises 1-ethyl-3-(3-dimethylaminopropyl)carbodiimide (EDC), N,N′-diisopropylcarbodiimide (DIC), hydroxybenzotriazole (HOBt), 3-[B is(dimethylamino)methyliumyl]-3H-benzotriazol-1-oxide hexafluorophosphate (HBTU), (Benzotriazol-1-yloxy)tripyrrolidinophosphonium hexafluorophosphate (PyBOP), bromotripyrrolidinophosphonium hexafluorophosphate (PyBrOP), or a combination of any of the foregoing. In some embodiments, the one or more additives comprises a base, for example, an organic base such as trimethylamine (TEA) or diisopropylethylamine (DIPEA).

In some embodiments, the coupling reactions are carried out at about 0° C. to about 60° C., for example, about 0° C. to about 20° C., about 10° C. to about 30° C., about 20° C. to about 40° C., about 30° C. to about 50° C., about 40° C. to about 60° C., about 0° C., about 5° C., about 10° C., about 15° C., about 20° C., about 25° C. (i.e., “room temperature”), about 30° C., about 35° C., about 40° C., about 45° C., about 50° C., about 55° C., about 60° C., or any value in between. In some embodiments, the coupling reactions are carried out at room temperature (about 25° C.). In some embodiments, the coupling reactions are carried out at about 37° C.

Each alignment bead can be modified with one or more different oligonucleotide binding sites using the chemical coupling reactions and conditions described above. The oligonucleotide sequence of each binding site is designed to be the same as the nucleic acid (gene) sequence of interest in the biological analytes and reverse-complementary to the targeting domains of the probes. Design of the oligonucleotide binding site sequences can be performed using a publicly-available online sequence calculator. In one example the online calculator can be accessed via the internet website: http://reverse-complement.com. For optimal hybridization efficiency, the length of the oligonucleotides is about 15-35 residues. In some embodiments, the length of the binding site is 33 oligonucleotide residues.

In some embodiments, the sample is from a human subject. In some embodiments, the sample is from a solid or liquid biopsy comprising cells from a human subject. In some embodiments, the subject has been previously diagnosed with cancer, for example, via the use of a regulatory agency-approved assay or diagnostic kit (such as an FDA-approved assay or diagnostic kit). In some embodiments, the subject is suspected of having cancer. In some embodiments, the subject is currently suffering from cancer. In some embodiments, the subject has previously suffered from cancer, but is not currently diagnosed with cancer. In some embodiments, the cells are cancer cells. In some embodiments, the cancer is known to be driven by one or more specific genetic mutations, including, but not limited to Brca1, Brca2, Ras, c-Myc, Bcr-Ab1, Trk, ErbB, Egfr, Raf, Pdgfr, Mek, c-Jun, Bcl-2.

### Methods for Using Alignment Beads in a Singleplexed or Multiplexed FISH Assay

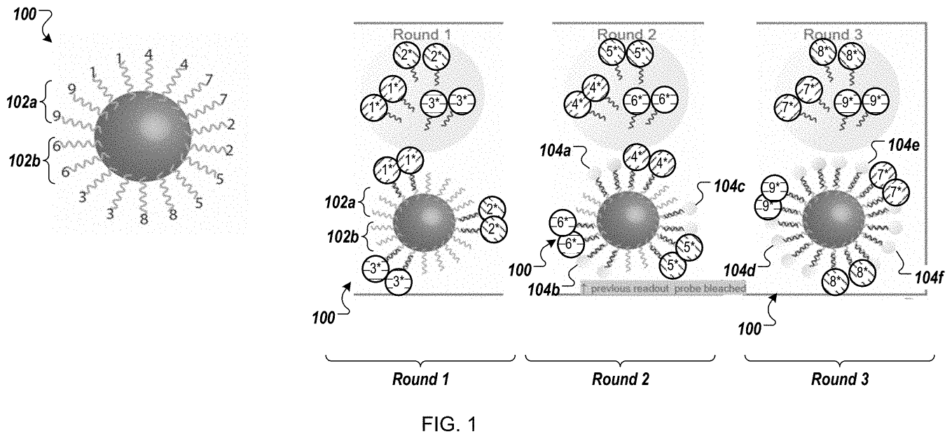

As described above, since the alignment beads (fiducial markers) can be functionalized with multiple different oligonucleotide binding sites, the alignment beads can be labeled with different readout-probes having with a single fluorophore (singleplex) or readout-probes having multiple different fluorophores (multiplex) for use in each round of hybridization and imaging. FIG. 1 depicts an alignment bead 100 functionalized with nine different binding sites 102a-b. The binding sites on the alignment bead encode reverse-complementary oligonucleotide sequences to the targeting domains of probes, which are conjugated to different color dyes. Three different probes are labeled with one dye (e.g., sequences 1, 4 and 7 are labeled with red dye, sequences 2, 5 and 8 are labeled with green dye, and sequences 3, 6 and 9 are labeled with yellow dye), and probes labeled in one or more colors can be used in a single hybridization round. For example, in Round 1, probes 1-3 are used; in Round 2, probes 4-6 are used; in Round 3, probes 7-9 are used. The sample is photo-bleached after imaging and registration in each round of hybridization, resulting on one or more bleached readout probes 104a-f during various rounds.

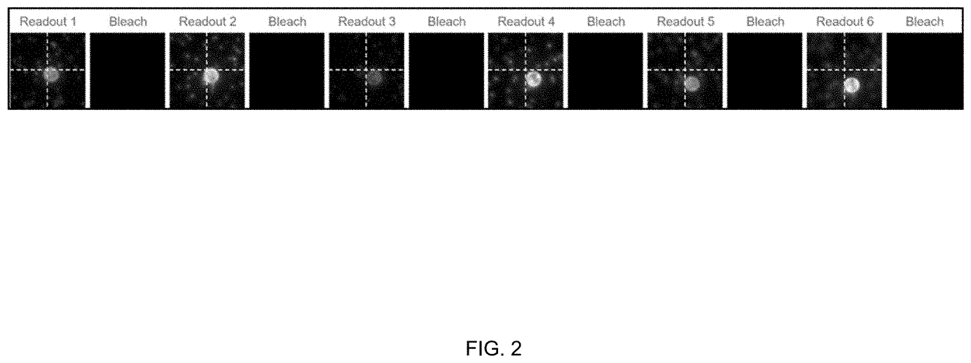

In one example of a single-color FISH experiment, six different single-color probes can be used to label the alignment beads in each round of hybridization, as shown in FIG. 2. Alignment beads of 1-μm diameter and functionalized with oligonucleotide binding sites complementary to the six probes, each of a single color, are used as fiducial markers. The same field of view is imaged throughout the rounds of hybridization. Six rounds of hybridization were performed, each round comprising (a) staining a sample with an aqueous buffer solution containing the alignment beads and DAPI; (b) hybridizing one type of single-color probes, each probe comprising an oligonucleotide targeting domain complementary to one oligonucleotide binding site of the alignment bead and a fluorescent dye; and (c) photo-bleaching the sample to remove the single-color probes at the end of each round. The 1-μm alignment beads show up in all rounds of imaging with different colors in each round. Offset of alignment beads between rounds is used to correct for image registration. In general, a minimum of 3 beads per FOV is sufficient to correct for shifts in the focus plane in between readout rounds.

An mFISH apparatus can correct for image registration between rounds by comparing two images of a sample, each captured during a different round. When mFISH apparatus determines that the alignment beads depicted in each of the two images are in approximately the same location within the images, e.g., are within a threshold distance of each other, e.g., less than 100 pixels the mFISH apparatus can determine to skip registration. When the mFISH apparatus determines that the locations of the alignment beads depicted in each of the two images are more than a threshold distance apart, the mFISH apparatus can determine a registration transformation for the image, e.g., a translation, rotation, or combination thereof, that that when applied to the image will move the location of each bead depicted in a first image to a corresponding second location of the bead depicted in a second image. The mFISH apparatus can then use apply this transformation to the image to perform image registration.

The mFISH apparatus can correct for registration by adjusting a location of a sample depicted in one of the images using the translation. This can cause the depiction of the sample in both images to be in approximately the same location.

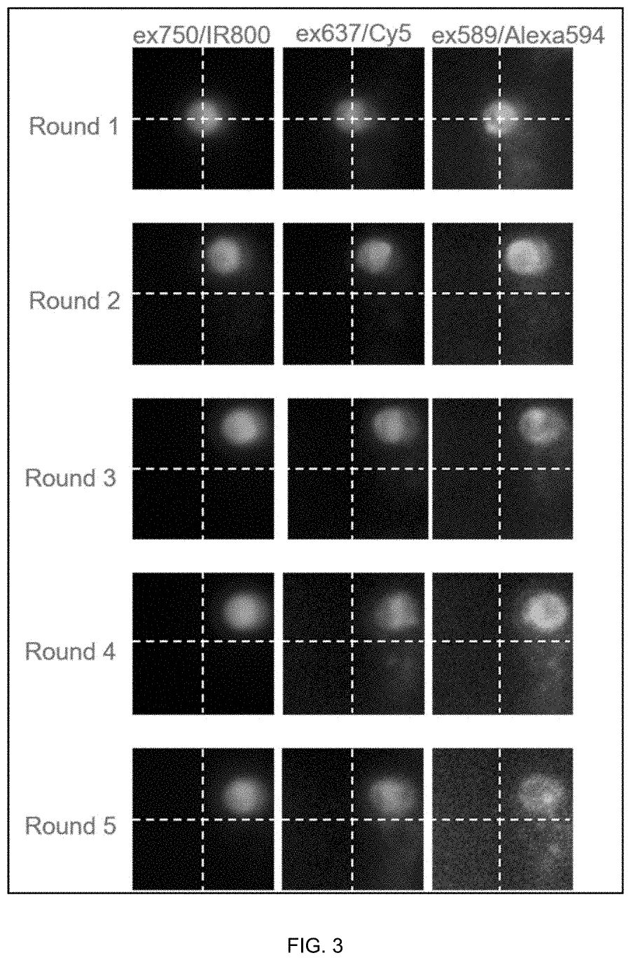

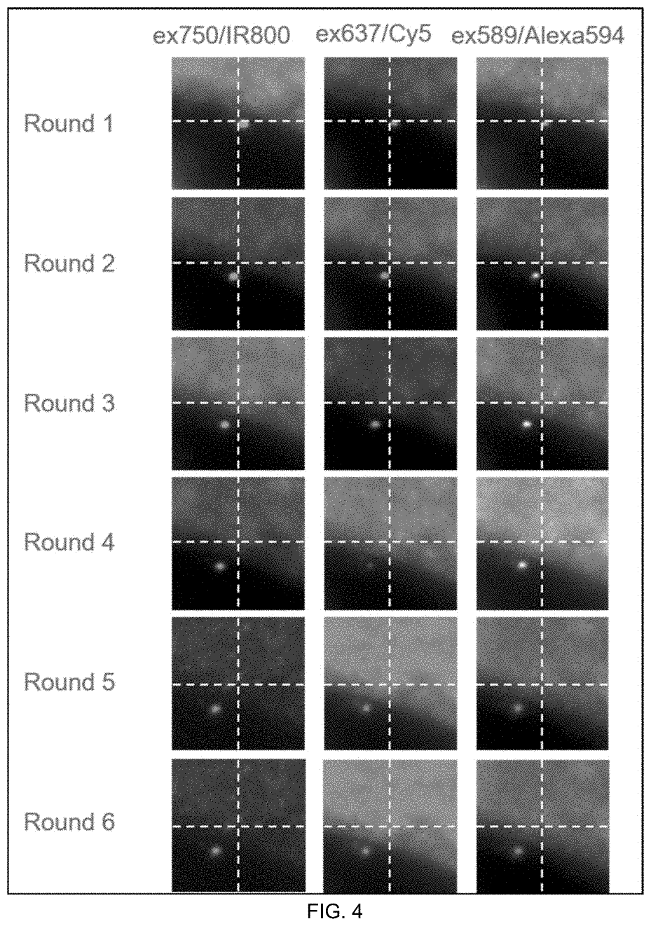

Samples can also be imaged with alignment beads labeled with multiple-color probes in a single round of hybridization. In one example of such multi-color FISH experiment, as shown in FIG. 3, fluorescent microscopy images were acquired sequentially of a 1-μm diameter alignment bead having multiple copies of fifteen different oligonucleotide binding sites attached to the bead surface. Five rounds of hybridization were performed on a sample with 1-μm diameter alignment bead embedded (adhered) on a glass, each round comprising (a) hybridizing three types of single-color probes, each probe comprising an oligonucleotide targeting domain complementary to one oligonucleotide binding site of the alignment bead and linked to a fluorescent dye (such as those described herein); (b) imaging the sample in three color channels; and (c) photo-bleaching the sample to remove the single-color probes in all three channels at the end of each round. The 1-μm alignment beads show up in all three fluorescence channels simultaneously during each round of hybridization and imaging. The tracking of three beads per FOV enables correction of shifts caused in between readout rounds. In another example, as shown in FIG. 4, fluorescent microscopy images were acquired sequentially of a 0.2-μm diameter alignment bead having multiple copies of eighteen different oligonucleotide binding sites attached to the bead surface. Six rounds of hybridization were performed on a sample with alignment bead embedded on a cell line sample, each round comprising (a) hybridizing three types of single-color probes, each probe comprising an oligonucleotide targeting domain complementary to one oligonucleotide binding site of the alignment bead and linked to a fluorescent dye (such as those described herein); (b) imaging the sample in three color channels; and (c) photo-bleaching the sample to remove the single-color probes in all three channels at the end of each round. The 0.2-μm alignment beads show up in all three fluorescence channels simultaneously during each round of hybridization and imaging. The tracking of three beads per FOV enables correction of shifts caused in between readout rounds.

### Methods for Correcting Focus Drift Using Oligonucleotide-Functionalized Alignment Beads as Fiducial Markers

In some embodiments, a fiducial identification system capable of registering fluorescence images captured at different time points is used in conjunction with the oligonucleotide-functionalized alignment beads as described in this specification. In one example, the fiducial identification system can be used to identify fiducial markers in a fluorescence microscopy image of a microscope slide containing fiducial markers and a sample having a common fluorescence color. The fluorescence microscopy image depicts both the fiducial markers and the sample. The fiducial markers identified in the image can be used to register the image to other images of the microscope slide taken at different time points. To register fluorescent images captured at different time points, the fiducial identification system is used to identify the positions of the fiducial markers depicted in the images, and the positions of the fiducial markers are used to determine the appropriate registration transformation.

The fiducial identification system is configured to process a first image acquired at a first time point and a second image acquired at a second time point to generate the respective fiducial marker positions. The fiducial marker positions for an image (e.g., the first image or the second image) define the positions of the fiducial markers depicted in the image. The position of a particular fiducial marker can be represented as a set of coordinates, for example, x-y coordinates in a frame of second of the image.

In one example, the contents of the microscope slide include a collection of ribonucleic acid (RNA) molecules (analytes of interest in a sample) and fiducial marker alignment beads, both labeled with the probes, are suspended in a microfluidic chamber. Between the first image and the second image being captured, the RNA molecules and the fiducial marker beads may have moved in the microfluidic chamber. In this example, the RNA molecules and the fiducial marker beads move approximately in unison (i.e., so the relative positions of RNA molecules and the fiducial marker beads remain approximately the same between time points). Since the position of the fiducial marker beads can be defined between the first and the second image, any shifts in the position of the RNA molecules can also be defined.

### EXAMPLES

The following materials and methods were used in the Examples set forth herein.

### Example 1. Functionalization of 0.2-μm Carboxyl Latex Beads with 5′-amino-oligonucleotides

A 5′-amino oligonucleotide, wherein the amino group is covalently linked to the 5′-phosphate group of the oligonucleotide with a 12-carbon spacer in between, can be procured from commercial suppliers. The oligonucleotides are prepared synthetically via conventional automated solid-state oligonucleotide synthesis, and the desired sequences can be specified as part of the oligonucleotide synthesis. The oligonucleotides can be supplied as dry powders, or as aqueous solutions in water or other aqueous buffers. The oligonucleotides as dry powders are preferred as they are more stable than when stored as an aqueous solution.

Oligonucleotides as dry powders were re-suspended in ultra-pure water to a final concentration of 1 mM, aliquoted into single-use vials and stored at -20° C. Frozen aliquots were thawed prior to use and were not re-frozen to avoid multiple freeze-thaw cycles. Upon thawing, 2.5-μL of the 1 mM (corresponding to 2.5 nmol) of each oligonucleotide solution was transferred to a 1.8-mL conical tube via an automatic micropipette. Multiple different oligonucleotide solutions can be combined in a single conical tube for each run of alignment bead functionalization reaction. To the combined oligonucleotides solution was added 1 volume of MES buffer; for example, if 16 oligonucleotide solutions were combined, 40-μL (15*2.5-μL) of the MES buffer would be added. The resulting solution was vortexed gently to mix the components.

1 μmol of a 4% (w/v) aqueous suspension of the 0.2-μm diameter carboxyl latex beads (Catalog no. C37486, Molecular Probes) was transferred to a 1.8-mL conical tube via a micropipette, to which 3 volumes of MES buffer was added, resulting in a 1% (w/v) bead suspension. The bead suspension was centrifuged at 12,000 g for 15 min to fully precipitate the beads. The supernatant was discarded, and the pellet was re-suspended in 400-μL of MES buffer with gentle vortexing. The resulting bead suspension was added to the oligonucleotide/MES solution prepared as described above.

A stock solution of 10 mg/mL EDC in ultrapure water was prepared and 5-μL (corresponding to about 250 nmol of EDC) of the stock solution was added to the bead/oligonucleotide mixture as described above to initiate the coupling reaction. The resulting mixture was vortexed at 200-1000 rpm at room temperature for 15 min, incubated at 4° C. for 3 hours, and then terminated by adding 50-μL of 1M Tris buffer to the reaction mixture followed by vortexing at 200-1000 rpm for 15 min. The mixture was then centrifuged at 12,000 g for 15 min, upon which the supernatant was discarded and the pellet re-suspended in 500-μL of TE buffer. The centrifugation/re-suspension steps were repeated once and the resulting oligonucleotide-functionalized beads were stored at refrigerated temperature (2-8° C.) until used.

### Example 2. Oligonucleotide Alignment Beads in a FISH Imaging Experiment

A 5-μL aliquot of the oligonucleotide-functionalized alignment beads, as prepared in Example 1, was mixed with 1 mL of each of a 1-10 μg/mL DAPI solution in PBS (in 1μg/mL increments), to target a 0.5-1% (v/v) bead/DAPI ratio. The resulting mixture was sonicated for 10 min, applied to a microscope slide. After 15 min, the sample was washed twice with 2× SSC, post-fixed with 4% PFA in PBS for 15 min, and then washed 3 times with 2× SSC. The sample containing alignment beads were subsequently imaged with the mFISH apparatus and the image capture process, described in more detail below.

### FISH Imaging System

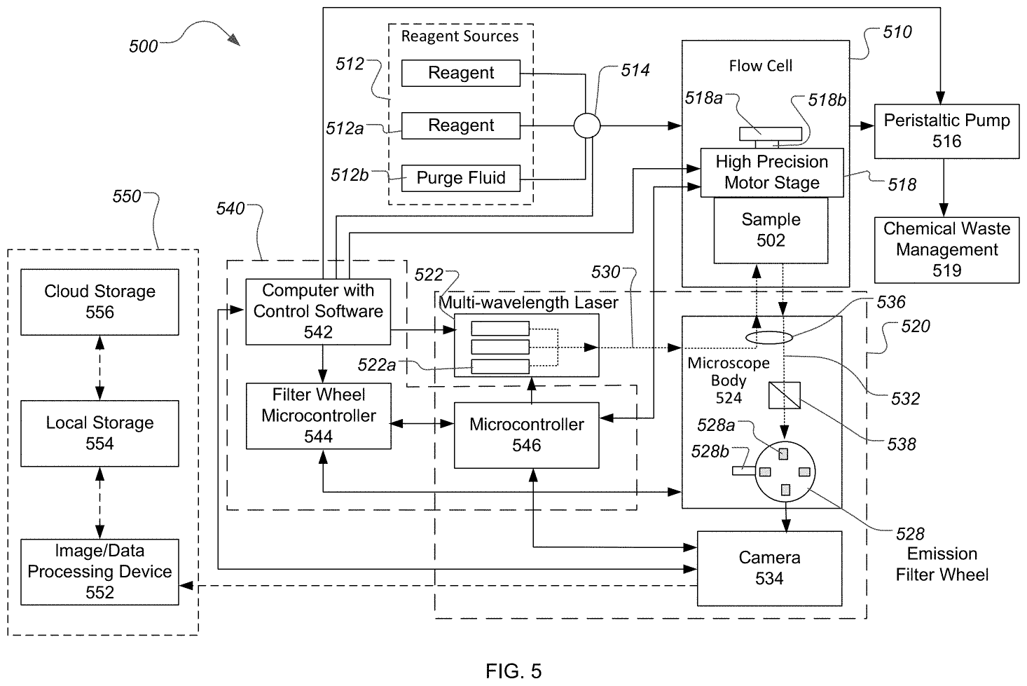

Referring to FIG. 5, a multiplexed fluorescent in-situ hybridization (mFISH) imaging and image processing apparatus 500 includes a flow cell 510 to hold a sample 502, a fluorescence microscope 520 to obtain images of the sample 502, and a control system 540 to control operation of the various components of the mFISH imaging and image processing apparatus 500. The control system 540 can include a computer 542, e.g., having a memory, processor, etc., that executes control software.

The fluorescence microscope 520 includes an excitation light source 522 that can generate excitation light 530 of multiple different wavelengths. In particular, the excitation light source 522 can generate narrow-bandwidth light beams having different wavelengths at different times. For example, the excitation light source 522 can be provided by a multi-wavelength continuous wave laser system, e.g., multiple laser modules 522a that can be independently activated to generate laser beams of different wavelengths. Output from the laser modules 522a can be multiplexed into a common light beam path.

The fluorescence microscope 520 includes a microscope body 524 that includes the various optical components to direct the excitation light from the light source 522 to the flow cell 510. For example, excitation light from the light source 522 can be coupled into a multimode fiber, refocused and expanded by a set of lenses, then directed into the sample 502 by a core imaging component, such as a high numerical aperture (NA) objective lens 536. When the excitation channel needs to be switched, one of the multiple laser modules 522a can be deactivated and another laser module 522a can be activated, with synchronization among the devices accomplished by one or more microcontrollers 544, 546.

The objective lens 536, or the entire microscope body 524, can be installed on vertically movable mount coupled to a Z-drive actuator. Adjustment of the Z-position, e.g., by a microcontroller 546 controlling the Z-drive actuator, can enable fine tuning of focal position. Alternatively, or in addition, the flow cell 510 (or a stage 518 supporting the sample in the flow cell 510) could be vertically movable by a Z-drive actuator 518b, e.g., an axial piezo stage. Such a piezo stage can permit precise and swift multi-plane image acquisition.

The sample 502 to be imaged is positioned in the flow cell 510. The flow cell 510 can be a chamber with cross-sectional area (parallel to the object or image plane of the microscope) with and area of about 2 cm by 2 cm. The sample 502 can be supported on a stage 518 within the flow cell, and the stage (or the entire flow cell) can be laterally movable, e.g., by a pair of linear actuators 518a to permit XY motion. This permits acquisition of images of the sample 502 in different laterally offset fields of view (FOVs). Alternatively, the microscope body 524 could be carried on a laterally movable stage.

An entrance to the flow cell 510 is connected to a set of hybridization reagents sources 512. A multi-valve positioner 514 can be controlled by the controller 540 to switch between sources to select which reagent 512a is supplied to the flow cell 510. Each reagent includes a different set of one or more oligonucleotide probes, e.g., readout probes. Each probe targets a different RNA sequence of interest, and has a different set of one or more fluorescent materials, e.g., phosphors, that are excited by different combinations of wavelengths. In addition to the reagents 512a, there can be a source of a purge fluid 512b, e.g., deionized (“DI”) water.

An exit to the flow cell 510 is connected to a pump 516, e.g., a peristaltic pump, which is also controlled by the controller 540 to control flow of liquid, e.g., the reagent or purge fluid, through the flow cell 510. Used solution from the flow cell 510 can be passed by the pump 516 to a chemical waste management subsystem 519.

In operation, the controller 540 causes the light source 522 to emit the excitation light 530, which causes fluorescence of fluorescent material in the sample 502, e.g., fluorescence of the probes that are bound to RNA in the sample and that are excited by the wavelength of the excitation light. The emitted fluorescent light 532, as well as back propagating excitation light, e.g., excitation light scattered from the sample, stage, etc., is collected by an objective lens 536 of the microscope body 524.

The collected light can be filtered by a multi-band dichroic mirror 538 in the microscope body 524 to separate the emitted fluorescent light from the back propagating illumination light, and the emitted fluorescent light is passed to a camera 534. The multi-band dichroic mirror 538 can include a pass band for each emission wavelength expected from the probes, e.g., the readout probes, under the variety of excitation wavelengths. Use of a single multi-band dichroic mirror (as compared to multiple dichroic mirrors or a movable dichroic mirror) can provide improved system stability.

The camera 534 can be a high resolution (e.g., 2048×2048 pixel) CMOS (e.g., a scientific CMOS) camera, and can be installed at the immediate image plane of the objective. Other camera types, e.g., CCD, may be possible. When triggered by a signal, e.g., from a microcontroller, image data from the camera can be captured, e.g., sent to an image processing system 550. Thus, the camera 534 can collect a sequence of images from the sample.

To further remove residual excitation light and minimize cross talk between excitation channels, each laser emission wavelength can be paired with a corresponding band-pass emission filter 528a. Each filter 528a can have a wavelength of 10-50 nm, e.g., 14-32 nm. In some implementations, a filter is narrower than the bandwidth of the fluorescent material of the probe resulting from the excitation, e.g., if the fluorescent material of the probe has a long trailing spectral profile.

The filters are installed on a high-speed filter wheel 528 that is rotatable by an actuator. The filter wheel 528 can be installed at the infinity space to minimize optical aberration in the imaging path. After passing the emission filter of the filter wheel 528, the cleaned fluorescence signals can be refocused by a tube lens and captured by the camera 534. The dichroic mirror 538 can be positioned in the light path between the objective lens 538 and the filter wheel 528.

To facilitate high speed, synchronized operation of the system, the control system 540 can include two microcontrollers 544, 546 that are employed to send trigger signals, e.g., TTL signals, to the components of the fluorescence microscope 520 in a coordinated manner. The first microcontroller 544 is directly run by the computer 542, and triggers actuator 528b of the filter wheel 528 to switch emission filters 528a at different color channels. The first microcontroller 544 or the computer 542 can trigger the second microcontroller 546, which sends digital signals to the light source 522 in order to control which wavelength of light is passed to the sample 502. For example, the second microcontroller 546 can send on/off signals to the individual laser modules of the light source 522 to control which laser module is active, and thus control which wavelength of light is used for the excitation light. After completion of switching to a new excitation channel, the second microcontroller 546 controls the motor for the piezo stage 518b to select the imaging height. Finally the second microcontroller 546 sends a trigger signal to the camera 534 for image acquisition.

Communication between the computer 542 and the device components of the mFISH apparatus 500 is coordinated by the control software. This control software can integrate drivers of all the device components into a single framework, and thus can allow a user to operate the imaging system as a single instrument (instead of having to separately control many devices).

The control software supports interactive operations of the microscope and instant visualization of imaging results. In addition, the control software can provide a programming interface which allows users to design and automate their imaging workflow. A set of default workflow scripts can be designated in the scripting language.

In some implementations, the control system 540 is configured, i.e., by the control software and/or the workflow script, to acquire fluorescence images (also termed simply “collected images” or simply “images”) in loops in the following order (from innermost loop to outermost loop): z-axis, color channel, lateral position, and reagent.

These loops may be represented by the pseudocode in Table 1, below.

TABLE 1
example control system loop pseudocode

```
for h = 1:N_hybridization
  % multiple hybridizations
  for f = 1:N_FOVs
    % multiple lateral field-of-views
    for c = 1:N_channels
      % multiple color channels
      for z = 1:N_planes
        % multiple z planes
        Acquire image(h, f, c, z);
      end % end for z
    end % end for c
  end % end for f
end % end for h
```

For the z-axis loop, the control system 540 causes the stage 518 to step through multiple vertical positions. Because the vertical position of the stage 518 is controlled by a piezoelectric actuator, the time required to adjust positions is small and each step in this loop can be extremely fast.

First, the sample can be sufficiently thick, e.g., a few microns, that multiple image planes through the sample may be desirable. For example, multiple layers of cells can be present, or even within a cell there may be a vertical variation in gene expression. Moreover, for thin samples, the vertical position of the focal plane may not be known in advance, e.g., due to thermal drift. In addition, the sample 502 may vertically drift within the flow cell 510. Imaging at multiple Z-axis positions can ensure most of the cells in a thick sample are covered, and can help identify the best focal position in a thin sample.

For the color channel loop, the control system 540 causes the light source 522 to step through different wavelengths of excitation light. For example, one of the laser modules is activated, the other laser modules are deactivated, and the emission filter wheel 528 is rotated to bring the appropriate filter into the optical path of the light between the sample 502 and the camera 534.

For the lateral position, the control system 540 causes the light source 522 to step through different lateral positions in order to obtain different fields of view (FOVs) of the sample. For example, at each step of the loop, the linear actuators supporting the stage 518 can be driven to shift the stage laterally. In some implementations, the control system 540 number of steps and lateral motion is selected such that the accumulated FOVs to cover the entire sample 502. In some implementations, the lateral motion is selected such that FOVs partially overlap.

For the reagent, the control system 540 causes the mFISH apparatus 500 to step through multiple different available reagents. For example, at each step of the loop, the control system 540 can control the valve 514 to connect the flow cell 510 to the purge fluid 512b, cause the pump 516 to draw the purge fluid through the cell for a first period of time to purge the current reagent, then control the valve 514 to connect the flow cell 510 to different new reagent, and then draw the new reagent through the cell for a second period of time sufficient for the probes in the new reagent to bind to the appropriate RNA sequences. Because some time is required to purge the flow cell and for the probes in the new reagent to bind, the time required to adjust reagents can be longer than the time required for other steps in the process, e.g., as compared to adjusting the lateral position, color channel or z-axis.

As a result, a fluorescence image is acquired for each combination of possible values for the z-axis, color channel (excitation wavelength), lateral FOV, and reagent. Because the innermost loop has the fastest adjustment time, and the successively surrounding loops are of successively slower adjustment time, this configuration can provide the most time efficient technique to acquire the images for the combination of values for these parameters.

A data processing system 550 is used to process the images and determine gene expression to generate the spatial transcriptomic data. At a minimum, the data processing system 550 includes a data processing device 552, e.g., one or more processors controlled by software stored on a computer readable medium, and a local storage device 554, e.g., non-volatile computer readable media, that receives the images acquired by the camera 534. For example, the data processing device 552 can be a work station with GPU processors or FPGA boards installed. The data processing system 550 can also be connected through a network to remote storage 556, e.g., through the Internet to cloud storage.

The data processing system 550 can process the images as described in more detail below. For instance, the data processing system 550 can perform one or more steps to stich images from different FOVs together.

In some implementations, the data processing system 550 performs on-the-fly image processing as the images are received. In particular, while data acquisition is in progress, the data processing device 552 can perform image pre-processing steps, such as filtering and deconvolution, that can be performed on the image data in the storage device 554 but which do not require the entire data set. Because filtering and deconvolution can be a major bottleneck in the data processing pipeline, pre-processing as image acquisition is occurring can significantly shorten the offline processing time and thus improve the throughput.

### Image Capture Process

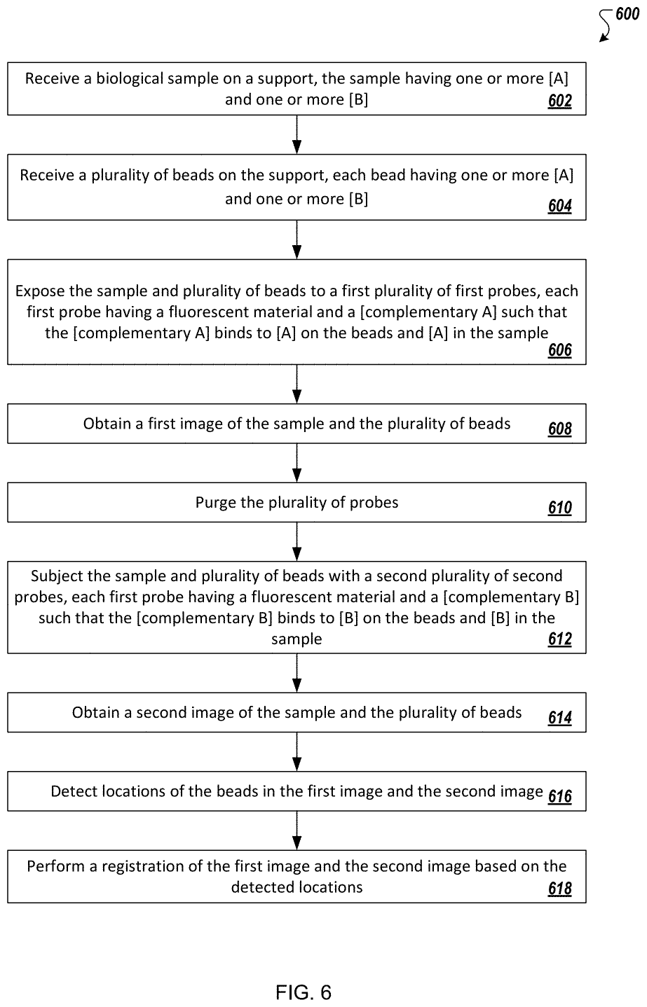

FIG. 6 is a flow diagram of a process 600 for registering a first image and a second image. For example, the process 600 can be used by the mFISH apparatus 500, e.g., an mFISH system, described with reference to FIG. 5.

An mFISH system receives a biological sample on a support (602). An operator can place the sample on the support in the mFISH system. For example, mFISH system receives the sample 502 on the flow cell 510, as described above. The sample, e.g., as part of RNA or DNA, (or a first targeting probe, e.g., as part of a readout portion of the targeting probe, that binds to the sample) has a first nucleotide sequence. The sample, e.g., as part of RNA or DNA, (or a second targeting probe, e.g., as part of a readout portion of the second targeting probe, that binds to the sample) has a second nucleotide sequence.

The mFISH system receives a plurality of beads on the support (604). Each bead has a plurality of binding sites. A first subset of the plurality of binding sites include the first nucleotide sequence. A second subset of the plurality of binding sites include the second nucleotide sequence. The beads can be fiducial markers as described in more detail above. For instance, each bead in the plurality of beads can be an alignment bead such as the alignment bead 100 described with reference to FIG. 1.

The mFISH system exposes the sample and plurality of beads to a first plurality of first probes (606). Each first probe has a fluorescent material and a complementary first nucleotide sequence such that the complementary first nucleotide sequence binds to the first nucleotide sequence on the beads and the first nucleotide sequence in the sample or in the targeting probe. For example, the mFISH system can expose the sample and the plurality of beads to the first plurality of first readout probes. The probes can be readout probes such as those described in more detail above.

In some examples, each of the first probes binds to either one of the beads or the sample. In some implementations, only a subset of the first probes binds to one of the beads or the sample. In these implementations, some of the first probes might not bind to a bead or the sample.

The mFISH system obtains a first image of the sample and the plurality of beads (608). For instance, the mFISH system positions the sample, and the support, at a first location and obtains the first image using a camera as described in more detail above. The mFISH system then obtains the first image that depicts both the sample and the plurality of beads. The first image can have a first z-axis location, a first color channel, and a first lateral position. The first lateral position can include a first x-axis location, and a first y-axis location.

The mFISH system can obtain different images using different color channels, as described in more detail above. For instance, the mFISH system can obtain the first image at a first color channel from multiple color channels and a second image at a second different color channel.

As part of the process to obtain an image, the mFISH system can excite fluorophores in the probes. The mFISH system can excite a fluorophore by sending a signal, generated by an excitation source such as a fluorescent microscopy apparatus, into the fluorophore that excites the fluorophore and causes the fluorophore to emit a fluorescent signal. The mFISH system can use a camera to obtain an image that depicts the fluorescent signals emitted by the fluorophores in the first probes.

The first image can depict data for all or a subset of the plurality of beads, all or a portion of the sample, or both. For instance, the first image can depict data for two or three of the beads and a portion of the sample.

The mFISH system purges the plurality of probes (610). For example, the mFISH system can use a purge fluid to purge the plurality of probes as described above. The purge process can purge all probes in the plurality of probes from the support. In some examples, the purge process removes some of the probes in the plurality of probes from the support, but not all probes in the plurality of probes. This can occur when the purge process removes probes from the support that have not bound to one of the beads or to the sample. In some implementations, instead of or as part of the purge process, the mFISH system can photobleach the sample, the beads, the probes, or a combination of two or more of these. For instance, the mFISH system can photobleach the sample, as described in more detail above. Photobleaching the sample can also cause photobleaching of the beads, the probes, or both, that are on the support.

The mFISH system subjects the sample and plurality of beads to a second plurality of second probes. Each second probe has a fluorescent material and a complementary second nucleotide sequence such that the complementary second nucleotide sequence binds to the second nucleotide sequence on the beads and the second nucleotide sequence in the sample or in the second targeting probes (612). The second probes are different probes from the first probes. The first plurality and the second plurality can be the same quantity or different quantities. The second probes can be readout probes, such as readout probes described in more detail above.

In some examples, each of the second probes binds to either one of the beads or the sample. In some implementations, only a subset of the second probes binds to one of the beads or the sample. In these implementations, some of the second probes might not bind to a bead or the sample.

The mFISH system obtains a second image of the sample and the plurality of beads (614). For example, the mFISH system uses the camera to obtain the second image. The mFISH system obtain the second image that depicts both the sample and the plurality of beads. The second image can depict data for all or a subset of the plurality of beads, all or a portion of the sample, or both. For instance, the second image can depict data for two or three of the beads and a portion of the sample.

The mFISH system obtain the second image using a second color channel. The second color channel can be the same color channel as the first color channel. The second color channel can be a different color channel as the first color channel.

The second image has a second z-axis location, and a second lateral position. The second lateral position has a second x-axis location and a second y-axis location. The second z-axis location can be the same z-axis location as the first z-axis location.

In some examples, when the lateral position is two-dimensional, the first lateral position and the second lateral position can overlap partially, e.g., for a stitching process, or completely, e.g., for a registration process. In these examples, the first image and the second image can both depict data for at least one of the plurality of beads. For example, the first image and the second image can both depict data for a first bead. The first image can depict data for two other beads that are not depicted in the second image. The second image can depict data for three other beads that are not depicted in the first image.

Both the first image and the second image can each depict different portions of the sample. The different portions of the sample can be overlapping while not being the exact same portion of the sample.

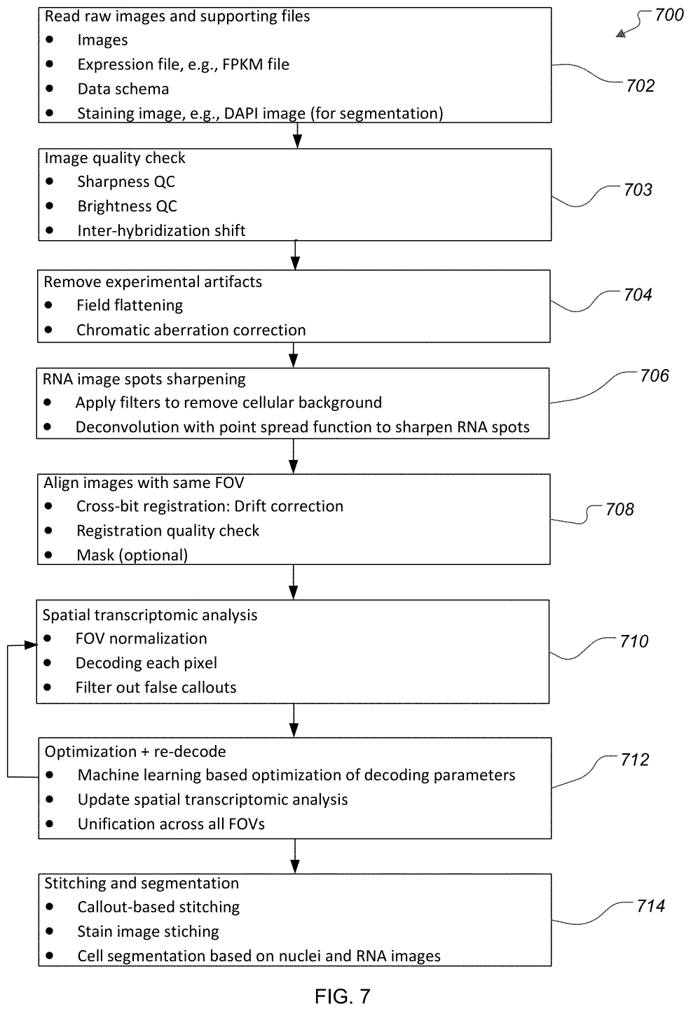

The mFISH system detects locations of the beads in the first image and the second image (616). For example, the mFISH system can detect the locations of the beads as described in more detail above and with reference to step 708 in FIG. 7.

Although the first image and the second image were captured at different lateral positions, and with different probes bound to the first bead, the mFISH system can detect the location of the first bead using data from the first image and the second image that represents the probes attached to the first bead. The mFISH system can determine that the first bead is depicted in both the first image and the second image using any appropriate process. For instance, the mFISH system can determine features depicted in each of the two images. The mFISH system can use properties of the depicted first bead, and features near the first bead that are depicted in both the first image and the second image, to determine that both images depict the same bead.

The mFISH system performs a registration of the first image and the second image based on the detected locations (618). For instance, the mFISH system can register the first image and the second image as described in more detail below, e.g., with reference to step 708 in FIG. 7 below.

The order of steps in the process 600 described above is illustrative only, and registering the first image and the second image can be performed in different orders. For example, the mFISH system can provide the plurality of beads on the support and then provide the biological sample on the support.

In some implementations, the process 600 can include additional steps, fewer steps, or some of the steps can be divided into multiple steps. For example, the mFISH system can perform one or more steps from the process 700 described with reference to FIG. 7 below.

### Image Stitching Process

FIG. 7 illustrates a flow chart of a process 700 of data processing in which the processing is performed after all of the images have been acquired. Although the process 700 is described as being performed after all images have been acquired, one or more steps in the process 700 can be performed before all images have been acquired. For instance, step 703, step 704, step 706, or a combination of these steps, can be performed for one or more first images while a data processing system, e.g., an image processing apparatus, continues to acquire one or more second images.

The process 700 begins with a data processing system receiving the raw image files and supporting files (step 702). In particular, the data processing system can receive the full set of raw images from the camera, e.g., an image for each combination of possible values for the z-axis, color channel (excitation wavelength), lateral FOV, and reagent.

The collected images can be subjected to one or more quality metrics (step 703) before more intensive processing in order to screen out images of insufficient quality. Depending on parameters for the data processing system, only images that meet the quality metric(s) can be passed on for further processing. This can significantly reduce processing load on the data processing system. For example, a sharpness quality value can be determined for each collected image to detect focusing failures As another example, in order to detect regions of interest, a brightness quality value can be determined for each collected image.

Next, some of the images can be processed to remove experimental artifacts (step 704). Since each RNA molecule will be hybridized multiple times with probes at different excitation channels, a strict alignment across the multi-channel, multi-round image stack can be beneficial for revealing RNA identities over the whole FOV. Removing the experimental artifacts can include field flattening and/or chromatic aberration correction. In some implementations, the field flattening is performed before the chromatic aberration correction.

One or more of the images can be processed to provide RNA image spot sharpening (step 706). RNA image spot sharpening can include applying filters to remove cellular background and/or deconvolution with point spread function to sharpen RNA spots.

The images having the same FOV are registered to align the features, e.g., the cells or cell organelles, therein (step 708). To accurately identify RNA species in the image sequences, features in different rounds of images are aligned, e.g., to sub-pixel precision. The images from the different rounds of images can each have a different color channel. For instance, one image can be captured at a first color channel for a first fluorescent material for the first probes used during that first round of imaging while another image can be captured at a second different color channel for a second different fluorescent material for the second probes used during that second round of imaging.

However, since an mFISH sample is imaged in aqueous phase and moved around by a motorized stage, sample drifts and stage drifts through hours-long imaging process can transform into image feature shifts, which can undermine the transcriptomic analysis if left unaddressed. In other words, even assuming precise repeatable alignment of the fluorescence microscope to the flow cell or support, the sample may no longer be in the same location in the later image, which can introduce errors into decoding or simply make decoding impossible.

The data processing apparatus can register images by placing fiducial markers, e.g., fluorescent beads, within the carrier material on the slide. In general, the sample and the fiducial marker beads will move approximately in unison. The data processing apparatus can identify these beads in the image based on their size and shape. Comparison of the positions of the beads can enable the data processing apparatus to register the two images, e.g., calculate of an affine transformation between the two images. As part of this process, the data processing apparatus can use features for the beads, for the portions of the sample surrounding the beads, or both, to detect beads that are depicted in multiple images. The multiple images can be images captured during different processing rounds. The data processing apparatus can then use these features to determine the images that depict the same bead. The data processing apparatus can then register the images that depict the same bead, e.g., as described in more detail above.

Optionally, after registration, a mask can be calculated for each collected image. In brief, the intensity value for each pixel is compared to a threshold value. A corresponding pixel in the mask is set to 1 if the intensity value is above the threshold, and set to 0 if the intensity value is below the threshold. The threshold value can be an empirically determined value, e.g., predetermined value, or can be calculated from the intensity values in the image. In general, the mask can correspond to the location of cells within the sample; spaces between cells should not fluoresce and should have a low intensity.

After registration of the images in a FOV, spatial transcriptomic analysis can be performed (step 710).

FOV normalization can be performed before the spatial transcriptomic analysis in order to make the histogram more consistent. In some implementations, the FOV normalization occurs after registration. Alternatively, FOV normalization can occur before registration. FOV normalization could be considered part of the filtering.

After normalization, an image stack can be evaluated as a 2-D matrix of pixel words as part of a process to decode each pixel. The matrix can have P rows, where P=X*Y, and B columns, where B is the number of images in the stack for a given FOV, e.g., N_hybridization*N_channels. Each row corresponds to one of the pixels (the same pixel across the multiple images in the stack), the values from the row provide a pixel word. Each column provides one of the values in the word, i.e., the intensity value from the image layer for that pixel. The values can be normalized, e.g., vary between 0 and I_{MAX}. I_{MAX} can have a value of 1.

If all the pixels are passed to the decoding step, then all P words will be processed as described below. However, pixels outside cell boundaries can be screened out by the 2-D masks and not processed. As result, computational load can be significantly reduced in the following analysis.

The data processing system 550 can store a code book that is used to decode the image data to identify the gene expressed at the particular pixel. The code book can include multiple reference code words, each reference code word associated with a particular gene. The code book can be represented as a 2D matrix with G rows, where G is the number of code words, e.g., the number of genes (although the same gene could be represented by multiple code words), and B columns. Each row can correspond to one of the reference code words, and each column can provide one of the values in the reference code word, as established by prior calibration and testing of known genes. For each column, the values in the reference code can be binary, i.e., “on” or “off”. For example, each value can be either 0 or I_{MAX}, e.g., 1.

For each pixel to be decoded, a distance d(p,i) is calculated between the pixel word and each reference code word. For example, the distance between the pixel word and reference code word can be calculated as a Euclidean distance, e.g., a sum of squared differences between each value in the pixel word and the corresponding value in the reference code word. This calculation can be expressed as:

$$d ( p , i ) = {\sum }_{x = 1}^{B} {( {I}_{p , x} - {C}_{i , x} )}^{2}$$

where I_{p,x} are the values from the matrix of pixel words and C_{i,x} are the values from the matrix of reference code words. Other metrics, e.g., sum of absolute value of differences, cosine angle, correlation, etc., can be used instead of a Euclidean distance.

Once the distance values for each code word are calculated for a given pixel, the smallest distance value is determined, the code word that provides that smallest distance value is selected as the best matching code word. Stated differently, the data processing apparatus determines min (d(p,1), d(p,2), . . . d(p,B)), and determines the value b as the value for i (between 1 and B) that provided the minimum. The gene corresponding to that best matching code word is determined, e.g., from a lookup table that associates code words with genes, and the pixel is tagged as expressing the gene.

The data processing apparatus can filter out false callouts. One technique to filter out false callouts is to discard tags where the distance value d(p,b) that indicated expression of a gene is greater than a threshold value, e.g., if d(p,b)>D1_{MAX}.

Yet another technique for filtering false callouts is to reject code words where a calculated bit ratio BR falls below a threshold. The bit ratio is calculated as the mean of the intensity values from the image word for layers that are supposed to be on (as determined from the code word), divided by the mean of the intensity values from the image word for layers that are supposed to be off (again as determined from the code word).

The bit ratio BR is compared to a threshold value THBR. In some implementations, the threshold value THBR is determined empirically from prior measurements. However, in some implementations, the threshold value THBR can be calculated automatically for a particular code word based on the measurements obtained from the sample.

Yet another technique for filtering false callouts is to reject code words where a calculated bit brightness BB falls below a threshold. The bit brightness is calculated as the mean of the intensity values from the image word for layers that are supposed to be on (as determined from the code word).

The bit brightness BB is compared to a threshold value TH_{BB}. In some implementations, the threshold value TH_{BB} is determined empirically from prior measurements. However, in some implementations, the threshold value TH_{BB} can be calculated automatically for a particular code word based on the measurements obtained from the sample.

The data processing apparatus can perform optimization and re-decoding (step 712). The optimization can include machine-learning based optimization of the decoding parameters, followed by updating spatial transcriptomic analysis using updated decoding parameters. This cycle can be repeated until the decoding parameters have stabilized.

The optimization of the decoding parameters can use a merit function, e.g., a FPKM/TPM correlation, spatial correlation, or confidence ratio. Parameters that can be included as variables in the merit function include the shape (e.g., start and end of frequency range, etc.) of the filters used to remove cellular background, the numerical aperture value for the point spread function used to sharpen the RNA spots, the quantile boundary Q used in normalization of the FOV, the bit ratio threshold TH_{BR}, the bit brightness threshold TH_{BB} (or the quantiles used to determine the bit ratio threshold TH_{BR} and bit brightness threshold TH_{BB}), and/or the maximum distance D1ₘₐₓ at which at which a pixel word can be considered to match a code word.

This merit function may be an effectively discontinuous function, so a conventional gradient following algorithm may be insufficient to identify the optimal parameter values. A machine learning model can be used to converge on parameter values.

Next, the data processing apparatus can perform unification of the parameter values across all FOVs. Because each FOV is processed individually, each field can experience different normalization, thresholding, filtering setting, or a combination of two or more of these. As a result, a high contrast image can result in a histogram with variation that causes false positive callouts in quiet areas. The result of unification is that all FOVs use the same parameter values. This can significantly remove callouts from background noise in quiet area, and can provide a clear and unbiased spatial pattern in large sample area.

A variety of approaches are possible to select a parameter value that will be used across all FOVs. One option is to simply pick a predetermined FOV, e.g., the first measured FOV or a FOV near the center of the sample, and use the parameter value for that predetermined FOV. Another option is to average the values for the parameter across multiple FOV and then use the averaged value. Another option is to determine which FOV resulted in the best fit between its pixel words and tagged code words. For example, a FOV with the smallest average distance d(p,b1) between the tagged code words and the pixel words for those code words can be determined and then selected.

The data processing apparatus can perform stitching and segmentation (step 714). Stitching combines multiple FOVs into a single image. Stitching can be performed using a variety of techniques. One approach is, for each row of FOV that together will form the combined image of the sample and each FOV within the row, determine a horizontal shift for each FOV. Once the horizontal shifting is calculated, a vertical shift is calculated for each row of FOV. The horizontal and vertical shifts can be calculated based on cross-correlation, e.g., phase correlation. With the horizontal and vertical shift for each FOV, a single combined image can be generated, and gene coordinates can be transferred to the combined image based on the horizontal and vertical shift.

An indication that a gene is expressed at a certain coordinate in the combined fluorescence image (as determined from the coordinate in the FOV and the horizontal and vertical shift for that FOV) can be added, e.g., as metadata. This indication can be termed a “callout.”

The stain images, e.g., the DAPI images, can be stitched together to generate a combined stain image. In some implementations, it is not necessary to create a combined fluorescence image from the collected fluorescence images; once the horizontal and vertical shift for each FOV is determined, the gene coordinates within the combined stain image can be calculated. The stain image can be registered to the collected fluorescent image(s). An indication that a gene is expressed at a certain coordinate in the combined stain image (as determined from the coordinate in the FOV and the horizontal and vertical shift for that FOV) can be added, e.g., as metadata, to provide a callout.

A potential problem remains in the stitched image. In particular, some genes may be double-counted in the overlapping area. To remove double-counting, a distance, e.g., Euclidean distance, can be calculated between each pixel tagged as expressing a gene and other nearby pixels tagged as expressing the same gene. One of the callouts can be removed if the distance is below a threshold value. More complex techniques can be used if a cluster of pixels are tagged as expressing a gene.

Segmentation of the combined image, e.g., the image of the stained cell, into regions corresponding to cells can be performed using various known techniques. Segmentation is typically performed after stitching of the images, but can occur before or after callouts are added to the combined image.

The segmented image with callouts indicating positions of gene expression, can now be stored and presented to a user, e.g., on a visual display, for analysis. Although the discussion above assumes that a single z-axis image is used in for each FOV, this is not required. Images from different z-axis positions can be processed separately; effectively the different z-axis positions provide a new set of FOVs.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network. The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination.

Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results . As one example, the processes depicted in the accompanying figures do not necessarily require the particular order shown, or sequential order, to achieve desirable results. In some cases, multitasking and parallel processing may be advantageous.

What is claimed is:

1. A method for determining the relative location of a biological analyte within a biological sample, the method comprising:
receiving a biological sample on a support, the sample having a first nucleotide sequence and a second nucleotide sequence or having a first and second pluralities of targeting probes that bind to targeted nucleotide sequences on the sample with the first plurality of targeting probes having the first nucleotide sequence and the second plurality of targeting probes having the second nucleotide sequence;
receiving a plurality of beads on the support, each bead having a plurality of binding sites, a first subset of the plurality of binding sites including the first nucleotide sequence and a second subset of the plurality of binding sites the second nucleotide sequence;
exposing the sample and plurality of beads to a first plurality of first probes, each first probe having a fluorophore and a complementary first nucleotide sequence such that the complementary first nucleotide sequence binds to the first nucleotide sequences on the beads and first nucleotide sequences in the sample or in the first plurality of targeting probes;
obtaining a first image of the sample and the plurality of beads;
deactivating the first plurality of first probes;
subjecting the sample and plurality of beads with a second plurality of second probes, each second probe having a fluorophore and a complementary second nucleotide sequence such that the complementary second nucleotide sequence binds to the second nucleotide sequence on the beads and the second nucleotide sequence in the sample or in the second plurality of targeting probes;
obtaining a second image of the sample and the plurality of beads;
detecting locations of the plurality of beads in the first image and the second image; and
performing a registration of the first image and the second image based on the detected locations.

2. The method of claim 1, comprising, for each of a plurality of pixels in the registered images, determining expression of genes in the sample based on intensities of fluorescence of the sample in the first image and the second image.

3. The method of claim 1, wherein deactivating the first plurality of first probes comprises photobleaching the first plurality of first probes.

4. The method of claim 3, wherein deactivating the first plurality of first probes comprises purging the first plurality of first probes.

5. The method of claim 1, wherein the first image and the second image have the same lateral position.

6. The method of claim 1, wherein the first image and the second image have the same color channel.

7. The method of claim 6, wherein obtaining the first image and obtaining the second image includes exciting the fluorophores in the first plurality of first probes and the second plurality of second probes with the same excitation wavelength.

8. The method of claim 7, wherein the fluorophores in the first plurality of first probes and the fluorophores of second plurality of second probes have the same material composition.

9. The method of claim 1, wherein the sample has a third nucleotide sequence and a fourth nucleotide sequence or has a third and fourth pluralities of targeting probes that bind to targeted nucleotide sequences on the sample with the third plurality of targeting probes having the third nucleotide sequence and the fourth plurality of targeting probes having the fourth nucleotide sequence.

10. The method of claim 9, comprising:
prior to deactivating the first plurality of first probes,
exposing the sample and plurality of beads to a third plurality of third probes, each third probe having a fluorophore and a complementary third nucleotide sequence such that the complementary third nucleotide sequence binds to the third nucleotide sequences on the beads and the third nucleotide sequences in the sample or in the third plurality of targeting probes, and
obtaining a third image of the sample and the plurality of beads.

11. The method of claim 10, wherein the first image and the third image use different color channels.

12. The method of claim 11, wherein fluorophores of the first plurality of first probes and fluorophores of the third plurality of third probes emit at different wavelengths.

13. The method of claim 12, wherein fluorophores of the first plurality of first probes and fluorophores of the third plurality of third probes are excited by different excitation wavelengths.

14. The method of claim 10, comprising detecting locations of the plurality of beads in the third image, and performing a registration of the first image and the third image based on the detected locations.

15. The method of claim 10, further comprising
deactivating the third plurality of third probes;
after deactivating the first plurality of first probes and the third plurality third probes,
subjecting the sample and plurality of beads with a fourth plurality of fourth probes, each fourth probe having a fluorophore and a complementary fourth nucleotide sequence such that the complementary fourth nucleotide sequence binds to the fourth nucleotide sequence on the beads and the fourth nucleotide sequence in the sample or in the fourth plurality of targeting probes; and
obtaining a fourth image of the sample and the plurality of beads.

16. The method of claim 15, wherein the second image and the fourth image use different color channels.

17. The method of claim 16, wherein the third image and the fourth image use the same color channel.

18. The method of claim 15, wherein fluorophores of the second plurality of second probes and fluorophores of the fourth plurality of fourth probes emit at different wavelengths and/or are excited by different excitation wavelengths..

19. The method of claim 18, wherein fluorophores of the third plurality of third probes and fluorophores of the fourth plurality of fourth probes emit at the same wavelength and/or have the same material composition and/or are excited by the same excitation wavelength.

20. The method of claim 15, comprising detecting locations of the plurality of beads in the fourth image, and performing a registration of the first image and the fourth image based on the detected locations.

21. A method for performing an in situ fluorescence hybridization assay on a sample, the method comprising:
a) contacting the sample with one or more targeting-probes, wherein each targeting-probe binds to an analyte in the sample, if present;
b) contacting the sample with a plurality of fiducial markers, wherein each fiducial marker comprises a plurality of binding sites;
c) contacting the sample with one or more readout-probes, wherein each readout-probe independently comprises a fluorescent moiety, and wherein each readout probe binds with the one or more targeting probes, if present, and the plurality of binding sites, thereby exhibiting one or more fluorescent signals;
d) imaging the one or more fluorescence signals produced by each readout-probe;
e) registering the image in step d);
f) Repeating steps (a)-(e) from 0 to 10 times;
g) Photobleaching the sample; and
h) Independently repeating steps (a)-(g) from 0 to 10 times.

22. The method of claim 21, wherein the sample is from a human subject.

23. The method of claim 22, wherein the subject has been previously diagnosed with cancer.

24. The method of claim 21, further comprising washing the sample between steps 1(a) and 1(d).

25. The method of claim 21, wherein for step (f), steps (a)-(e) are repeated from 1-9 times, thereby providing 2-10 total rounds of imaging.
